SEQUENCE LISTING (25 sequences)

SEQ ID NO: 1-8 - skipped (8 entries)

SEQ ID NO: 9 - DNA, 31 bp, Artificial Sequence
  Description of Artificial Sequence: Synthetic oligonucleotide
tcagagaacaacaacaagtggcttattttga

SEQ ID NO: 10 - DNA, 30 bp, Artificial Sequence
  Description of Artificial Sequence: Synthetic oligonucleotide
tcaaaataagccacttgttgttgttctctg

SEQ ID NO: 11 - DNA, 55 bp, Artificial Sequence
  Description of Artificial Sequence: Synthetic oligonucleotide
ataagtagcaagtggcgcataagtatcaaaataagccacttgttgttgttctctg

SEQ ID NO: 12 - DNA, 56 bp, Artificial Sequence
  Description of Artificial Sequence: Synthetic oligonucleotide
ccagagaacaacaacaagtggcttattttgatacttatgcgccacttgctacttat

SEQ ID NO: 13 - DNA, 38 bp, Artificial Sequence
  Description of Artificial Sequence: Synthetic oligonucleotide
tagtcccttaagcggagccctatagtgagtcgtattac

SEQ ID NO: 14 - DNA, 36 bp, Artificial Sequence
  Description of Artificial Sequence: Synthetic oligonucleotide
gtaatacgactcactatagggctccgcttaagggac

SEQ ID NO: 15 - DNA, 86 bp, Artificial Sequence
  Description of Artificial Sequence: Synthetic primer
aatgatacggcgaccaccgagatctacactctttccctacacgacgctcttccgatctgcgtcgcataagtatcaaaataagccac

SEQ ID NO: 16 - DNA, 56 bp, Artificial Sequence
  Description of Artificial Sequence: Synthetic primer
caagcagaagacggcatacgagctcttccgatctgtaatacgactcactatagggc

SEQ ID NO: 17 - DNA, 92 bp, Artificial Sequence
  Description of Artificial Sequence: Synthetic oligonucleotide
aatgatacggcgaccaccgagatctacactctttccctacacgacgctcttccgatctgcgttcaaaataagccacttgttgttgttctctg

SEQ ID NO: 18 - DNA, 93 bp, Artificial Sequence
  Description of Artificial Sequence: Synthetic oligonucleotide
ccagagaacaacaacaagtggcttattttgaacgcagatcggaagagcgtcgtgtagggaaagagtgtagatctcggtggtcgccgtatcatt

SEQ ID NO: 19 - DNA, 92 bp, Artificial Sequence
  Description of Artificial Sequence: Synthetic oligonucleotide
caagcagaagacggcatacgagctcacactctttccctacacgacgctcttccgatctgcgttcaaaataagccacttgttgttgttctctg

SEQ ID NO: 20 - DNA, 93 bp, Artificial Sequence
  Description of Artificial Sequence: Synthetic oligonucleotide
ccagagaacaacaacaagtggcttattttgaacgcagatcggaagagcgtcgtgtagggaaagagtgtgagctcgtatgccgtcttctgcttg

SEQ ID NO: 21 - protein, 612 aa, Artificial Sequence
  Description of Artificial Sequence: Synthetic polypeptide
MEKMDNLEVKAKINQGLYKITPRHKGTSFIWNVLADIQKEDDTLVEGWVFCRKCEKVLKYTTRQTSNLCRHKCCASLKQSRELKTVSADCKKEAIEKCAQWVVRDCRPFSAVSGSGFIDMIKFFIKVGAEYGEHVNVEELLPSPITLSRKVTSDAKEKKALISREIKSAVEKDGASATIDLWTDNYIKRNFLGVTLHYHENNELRDLILGLKSLDFERSTAENIYKKLKAIFSQFNVEDLSSIKFVTDRGANVVKSLANNIRINCSSHLLSNVLENSFEETPELNMPILACKNIVKYFKKANLQHRLRSSLKSECPTRWNSTYTMLRSILDNWESVIQILSEAGETQRIVHINKSIIQTMVNILDGFERIFKELQTCSSPSLCFVVPSILKVKEICSPDVGDVADIAKLKVNIIKNVRIIWEENLSIWHYTAFFFYPPALHMQQEKVAQIKEFCLSKMEDLELINRMSSFNELSATQLNQSDSNSHNSIDLTSHSKDISTTSFFFPQLTQNNSREPPVCPSDEFEFYRKEIVILSEDFKVMEWWNLNSKKYPKLSKLALSLLSIPASSAASERTFSLAGNIITEKRNRIGQQTVDSLLFLNSFYKNFCKLDI

SEQ ID NO: 22 - protein, 612 aa, Artificial Sequence
  Description of Artificial Sequence: Synthetic polypeptide
MEKMDNLEVKAKINQGLYKITPRHKGTSFIWNVLADIQKEDDTLVEGWVFCRKCEKVLKYTTRQTSNLCRHKCCASLKQSRELKTVSADCKKEAIEKCAQWVVRDCRPFSAVSGSGFIDMIKFFIKVGAEYGEHVNVEELLPSPITLSRKVTSDAKEKKALISREIKSAVEKDGASATIDLWTDNYIKRNFLGVTLHYHENNELRDLILGLKSLDFERSTAENIYKKLKAIFSQFNVEDLSSIKFVTDRGANVVKSLANNIRINCSSHLLSNVLENSFEETPELNMPILACKNIVKYFKKANLQHRLRSSLKSECPTRWNSTYTMLRSILDNWESVIQILSEAGETQRIVHINKSIIQTMVNILDGFERIFKELQTCSSPSLCFVVPSILKVKEICSPDVGDVADIAKLKVNIIKNVRIIWEENLSIWHYTAFFFYPPALHMQQEKVAQIKEFCLSKMEDLELINRMSSFNELSATQLNQSDSNSHNSIDLTSHSKDISTTSFFFPQLTQNNSREPPVSPSDEFEFYRKEIVILSEDFKVMEWWNLNSKKYPKLSKLALSLLSIPASSAASERTFSLAGNIITEKRNRIGQQTVDSLLFLNSFYKNFCKLDI

SEQ ID NO: 23 - DNA, 5908 bp, Artificial Sequence
  Description of Artificial Sequence: Synthetic polynucleotide
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggagaaaatggacaatttggaagtgaaagcaaaaatcaaccaaggattatataaaattactccgcgacataaaggaacaagttttatttggaacgttttagcggatatacagaaagaagacgatacattggtggaagggtgggtgttttgccgaaaatgcgaaaaagttttaaaatacacaactaggcagacatcaaacttatgtcgtcataaatgctgtgcctctctaaagcaatcccgagaattaaaaactgtttcagctgattgcaaaaaggaagcaattgaaaaatgtgcacaatgggtggtacgagattgtcggcctttttcggccgtctctggatccggctttatcgatatgataaaattttttattaaagttggagccgaatatggtgaacatgtcaacgttgaggaattgttaccaagtccaataacgctatcgagaaaggtaacttcggatgcaaaagaaaaaaaagctctgattagtcgagaaattaagtctgctgtagagaaagatggtgcatcagcaacgatagatttgtggaccgataattatataaaacggaattttttgggagtaacgttacactaccatgaaaacaatgaactgcgagatctaattttaggtttaaagtccttagattttgaaagatccacagcagaaaatatttataagaagcttaaagccattttttcacaattcaacgtcgaagacttgagtagtataaaatttgtgacagatagaggagccaatgtcgtaaaatcattggcaaataatatcagaattaactgcagcagccatttgctttcaaacgtgttggaaaattcatttgaggagacacctgaactcaatatgcctattcttgcttgcaaaaatattgtaaaatatttcaagaaagccaatctgcagcacagacttcgaagttctttaaaaagtgagtgccctacacggtggaattccacatacacgatgcttcgatctattctcgacaactgggaaagcgtgattcaaatattaagtgaggcgggagagacacagagaattgttcatataaataagtcgataattcaaacaatggtcaacatcctcgatgggtttgaaagaatttttaaagaattacaaacatgcagttcaccatctctgtgttttgttgtgccttccattttaaaagtaaaagaaatatgttcacctgacgttggcgacgttgcagatatagcaaaattgaaagtgaacattataaaaaatgtaagaataatatgggaagaaaatttaagcatatggcactacacagcattttttttctatccgcccgccttgcatatgcaacaagagaaagtggcacaaattaaagaattttgcttatccaaaatggaagatttggaattaataaaccgcatgagttcctttaacgaattatccgcaactcagcttaaccagtcggactccaatagccacaacagtatagatttaacatcccattcaaaagacatttcaacgacaagtttctttttcccgcaattaactcagaacaatagtcgtgagccaccagtgtgtccaagcgatgaatttgaattttatcgtaaagaaatagttattttaagcgaagattttaaagttatggaatggtggaatcttaattcaaaaaagtatcctaaactatctaaactggctttgtcgttattatcaatacctgcaagtagcgctgcatcggaaaggacattttccctagctggaaatataataactgaaaagagaaacaggattgggcaacaaactgtcgacagcttgttatttttaaattccttttacaaaaatttttgtaaattagatatatgactggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 24 - DNA, 5908 bp, Artificial Sequence
  Description of Artificial Sequence: Synthetic polynucleotide
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggagaaaatggacaatttggaagtgaaagcaaaaatcaaccaaggattatataaaattactccgcgacataaaggaacaagttttatttggaacgttttagcggatatacagaaagaagacgatacattggtggaagggtgggtgttttgccgaaaatgcgaaaaagttttaaaatacacaactaggcagacatcaaacttatgtcgtcataaatgctgtgcctctctaaagcaatcccgagaattaaaaactgtttcagctgattgcaaaaaggaagcaattgaaaaatgtgcacaatgggtggtacgagattgtcggcctttttcggccgtctctggatccggctttatcgatatgataaaattttttattaaagttggagccgaatatggtgaacatgtcaacgttgaggaattgttaccaagtccaataacgctatcgagaaaggtaacttcggatgcaaaagaaaaaaaagctctgattagtcgagaaattaagtctgctgtagagaaagatggtgcatcagcaacgatagatttgtggaccgataattatataaaacggaattttttgggagtaacgttacactaccatgaaaacaatgaactgcgagatctaattttaggtttaaagtccttagattttgaaagatccacagcagaaaatatttataagaagcttaaagccattttttcacaattcaacgtcgaagacttgagtagtataaaatttgtgacagatagaggagccaatgtcgtaaaatcattggcaaataatatcagaattaactgcagcagccatttgctttcaaacgtgttggaaaattcatttgaggagacacctgaactcaatatgcctattcttgcttgcaaaaatattgtaaaatatttcaagaaagccaatctgcagcacagacttcgaagttctttaaaaagtgagtgccctacacggtggaattccacatacacgatgcttcgatctattctcgacaactgggaaagcgtgattcaaatattaagtgaggcgggagagacacagagaattgttcatataaataagtcgataattcaaacaatggtcaacatcctcgatgggtttgaaagaatttttaaagaattacaaacatgcagttcaccatctctgtgttttgttgtgccttccattttaaaagtaaaagaaatatgttcacctgacgttggcgacgttgcagatatagcaaaattgaaagtgaacattataaaaaatgtaagaataatatgggaagaaaatttaagcatatggcactacacagcattttttttctatccgcccgccttgcatatgcaacaagagaaagtggcacaaattaaagaattttgcttatccaaaatggaagatttggaattaataaaccgcatgagttcctttaacgaattatccgcaactcagcttaaccagtcggactccaatagccacaacagtatagatttaacatcccattcaaaagacatttcaacgacaagtttctttttcccgcaattaactcagaacaatagtcgtgagccaccagtgagtccaagcgatgaatttgaattttatcgtaaagaaatagttattttaagcgaagattttaaagttatggaatggtggaatcttaattcaaaaaagtatcctaaactatctaaactggctttgtcgttattatcaatacctgcaagtagcgctgcatcggaaaggacattttccctagctggaaatataataactgaaaagagaaacaggattgggcaacaaactgtcgacagcttgttatttttaaattccttttacaaaaatttttgtaaattagatatatgactggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 25 - protein, 6 aa, Artificial Sequence
  Description of Artificial Sequence: Synthetic 6xHis tag
HHHHHH

(19) United States
(12) Patent Application Publication
(10) Pub. No.: US 2017/0114333 A1
(43) Pub. Date: Apr. 27, 2017

(54) IMPROVEMENTS TO EUKARYOTIC TRANSPOSASE MUTANTS AND TRANSPOSON END COMPOSITIONS FOR MODIFYING NUCLEIC ACIDS AND METHODS FOR PRODUCTION AND USE IN THE GENERATION OF SEQUENCING LIBRARIES

(71) Applicant: THE JOHNS HOPKINS UNIVERSITY, Baltimore, MD (US)

(72) Inventors: Nancy L. Craig, Baltimore, MD (US); Sunil Gangadharan, Baltimore, MD (US); Fred Dyda, Washington, DC (US); Alison Hickman, Washington, DC (US)

(21) Appl. No.: 15/302,570

(22) PCT Filed: Apr. 10, 2015
(86) PCT No.: PCT/US2015/025261

§ 371 Date: Oct. 7, 2016

Related U.S. Application Data
(60) Provisional application No. 61/978,498, filed on Apr. 11, 2014.

(51) Int. Cl. C12N 9/12; C12Q 1/68

(52) U.S. Cl. CPC C12N 9/1241; C12Y 207/07; C12Q 1/6874

(57) ABSTRACT

Hyperactive Hermes Transposase mutants and genes encoding them are disclosed. These transposases are easily purified in large quantity after expression in bacteria. The modified Hermes Transposases are soluble and stable and exist as smaller active complexes compared to the native enzyme. The consensus target DNA recognition sequence is the same as the native enzyme and shows minimal insertional sequence bias. These properties are useful in whole genome sequencing applications that involve sample DNA preparation requiring simultaneous fragmentation and attachment of custom sequences to the ends of the fragments. Methods and compositions using these transposases in fragmentation and 5′ end-tagging are also disclosed.

### RELATED APPLICATIONS

This application claims the benefit of priority under 35 U.S.C. §119(e) to U.S. Provisional Application No. 61/978,498, filed on Apr. 11, 2014, which is incorporated herein by reference in its entirety.

### U.S. GOVERNMENT SUPPORT

This invention was made with U.S. government support under grant number NIH GM076425. The U.S. government has certain rights in the invention.

### FIELD OF THE INVENTION

The current invention relates to mutated transposases and methods to use them for fragmenting and tagging target DNA for use in next generation DNA sequencing.

### DESCRIPTION OF THE BACKGROUND

Transposons, segments of DNA that can mobilize to other locations in a genome, are useful for insertion mutagenesis and for generation of priming sites for sequencing of DNA molecules. In vitro, transpositions using transposases and transposons can be used to generate mutagenized plasmid/fosmid libraries for large scale phenotypic screening. More recently, the ability of transposase and transposon end compositions to bring about fragmentation and 5′ tagging of DNA has been exploited in generating libraries of tagged DNA fragments for Next Generation sequencing platforms. Such applications for “cut and paste” DNA transposons Tn5 and Mu and the advantages of using them over methods involving mechanical fragmentation are disclosed in Published U.S. Patent Application 2011/0287435. For these uses, a transposon with minimal insertion bias is desired to allow complete coverage with minimal oversampling. Tn5 and Mu transposons show unfavorable insertional sequence bias. A modified Tn7 TnsABC-only system has low sequence bias but requires the expression and purification of several different subunits to form the active complex and is therefore cumbersome to exploit commercially. Moreover, the frequency of transposition is very low for most transposons and there is a requirement in the art for hyperactive transposases. The modified Hermes Transposase of the present invention is a substantial improvement for the above mentioned applications because of the combination of its higher activity and reduced insertional bias. Transposons have also been used in vivo in generating transgenic organisms as disclosed in Published U.S. Patent Application 2003/0150007. The modified form of Hermes Transposase can also be used for such in vivo applications. In vivo insertional mutagenesis methods using transposons in general e.g. Hermes is disclosed in Published U.S. Patent Application 2004/0092018. These patent applications are incorporated herein by reference to the extent permitted by applicable statute and regulation.

### SUMMARY OF THE INVENTION

Described herein are Hermes transposases and reaction conditions that result in increased strand transfer in vitro, thereby increasing the efficiency of nucleic acid modification. Also described herein is the use of the wild type version of Hermes that may also be modified by mutation to prevent aggregation at particular KCL concentrations. The transposase mutants described herein are used for the development of DNA libraries and next generation sequencing. The mutant transposases disclosed in this invention are a modified form of the native Hermes Transposase, have a similar mechanism of action as the wild type, can easily be expressed in the bacterium, E. coli, and purified in large quantities. The transposases also have the additional advantage of not requiring a preformed transposase complex as in existing alternative transposons such as Tn5 and Mu.6. The transposases described herein, unlike alternatives that have to be incubated at 37° C., is fully active at room temperature at 23° C. up to 30° C. so that the reaction can be readily carried out on a laboratory benchtop.

The modified Hermes Transposases of the invention, as a result of the introduced mutations are octameric. These Hermes Transposases also have a higher transposition activity in vitro than do the wild type transposase. Compared to existing commercialized transposases, the modified Hermes Transposases have less insertional sequence bias when used for in vitro fragmentation of genomic DNA and 5′ end tagging followed by next generation sequencing.

Described herein is an improved hyperactive mutant transposase having a sequence selected from the group consisting of SEQ ID NO: 21, SEQ ID NO: 22, SEQ ID NO: 23, and SEQ ID NO: 24.

The invention provides methods of fragmenting and tagging target DNA sequences comprising the steps of: providing ligand labeled Hermes LEs; reacting the labeled Hermes LEs with an improved mutant transposase and target DNA sequences whereby each target DNA sequence becomes fragmented and each DNA fragment is labeled at either end by one of the labeled Hermes LEs; purifying the labeled DNA fragments using an affinity system that binds the ligand.

In some cases, the affinity system employs beads that bind the ligand. For example, the beads are magnetic beads. The ligand is biotin or polyhistidine of at least six histidine residues and the affinity system is biotinstreptavidin or nickel or cobalt affinity material, respectively.

Optionally, the method further comprises using a DNA polymerase to fill in gaps. For example, the DNA polymerase is T4 polymerase.

In some cases, the method further comprises a step of enzymatically cutting the tagged DNA following the step of purifying to replace one of the labeled Hermes LEs on each fragment with a specific terminal sequence. For example, PCR, DNA ligase or DNA polymerase chain extension is used to add the specific terminal sequence.

In one aspect, the method further comprises the step of using a second transposon system to introduce a second tag into each DNA fragment. For example, the step of using a second transposon system follows the step of purifying. In some cases, the second transposon system is a piggy Bac transposon.

Also provided is a method of fragmenting and tagging target DNA sequences comprising the steps of: providing tagged Hermes LEs bearing at least one specific sequence tag; and reacting the tagged Hermes LEs with an improved mutant transposase and target DNA sequences whereby each target DNA sequence becomes fragmented and each DNA fragment is labeled at either end by one of the tagged Hermes LEs.

Optionally, the method further comprises the step of employing a DNA polymerase to fill in gaps. For example, the DNA polymerase is T4 polymerase.

In some cases, the method further comprises the step of using a second transposon system to introduce a second tag into each DNA fragment. For example, the second transposon system is a piggy Bac transposon. In one aspect, the method further comprises the step of enzymatically cutting the tagged DNA following the step of purifying to replace one of the tagged Hermes LEs on each fragment with a specific terminal sequence. For example, PCR, DNA ligase or DNA polymerase chain extension is used to add the specific terminal sequence.

Improved transposases that are hyperactive in vitro and useful for DNA fragmentation include: E55K, A290S, G366E, G366W, I370V, F433S, E459G, I498T, S499L, and T500M each of which being numbered in accordance with HermesI-612 wildtype (SEQ ID NO: 21).

Polynucleotides, polypeptides, or other agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W.H. Freeman and Company, incorporated herein by reference.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 illustrates WT, delta497-516, and Triple mutant polypeptide chains.

FIG. 2 shows the Hermes mechanism including excision and strand transfer.

FIG. 3 shows the modeled quaternary crystal structure of the wild type (WT) Hermes octamer.

FIG. 4 is a diagram showing the relationship between the wild type octamer and the mutated dimer interfaces.

FIG. 5 shows HIS6-peptide derivatized Hermes transposon end based fragmentation and tagging.

FIG. 6 is an agarose gel showing activity comparing WT and delta497-516, and Triple mutant Hermes transposases.

FIG. 7 is a diagram of the strand transfer reaction mediated by transposons.

FIG. 8 shows the a general scheme for transposase-based fragmentation and covalent tag attachment to the 5″ ends of target DNA.

FIG. 9 illustrates fragmentation of target DNA and 5′-tagging using a biotinylated Hermes LE and streptavidin beads.

FIG. 10 illustrates fragmentation and tagging using biotinylated Hermes LE, adding a second tag via a different transposase (piggy Bac) for PCR and high throughput sequencing.

FIG. 11 illustrates fragmentation and tagging using HIS6 peptide tagged Hermes LE oligonucleotides, purification with Ni NTA beads, DNA polymerase extension and strand displacement and final elution with imidazole.

FIG. 12 shows a photograph of an agarose gel showing the results of a strand transfer assay with Hermes wild type and a deletion dimer (KCL titration).

### DETAILED DESCRIPTION OF THE INVENTION

The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein specifically to provide improved embodiments of modified Hermes transposases.

Transposons are mobile genetic elements that are an important source of genetic variation and are useful tools for genome engineering, mutagenesis screens, and vectors for transgenesis including gene therapy.

For example, cell free systems for inter-molecular transposition for DNA sequencing, to create deletions or insertions into genes, and for studying protein domain functions have been developed for Tn7 (1), for Tn5 (2), and for Mu (3).

Hermes is a 2479 bp long hAT family DNA transposon element derived from the Maryland strain of the common housefly Musca domestica. Its use in creating transgenic insects was disclosed both in a research publication (4), and in U.S. Pat. No. 5,614,398, which is incorporated herein by reference to the extent permissible under applicable statute and regulation.

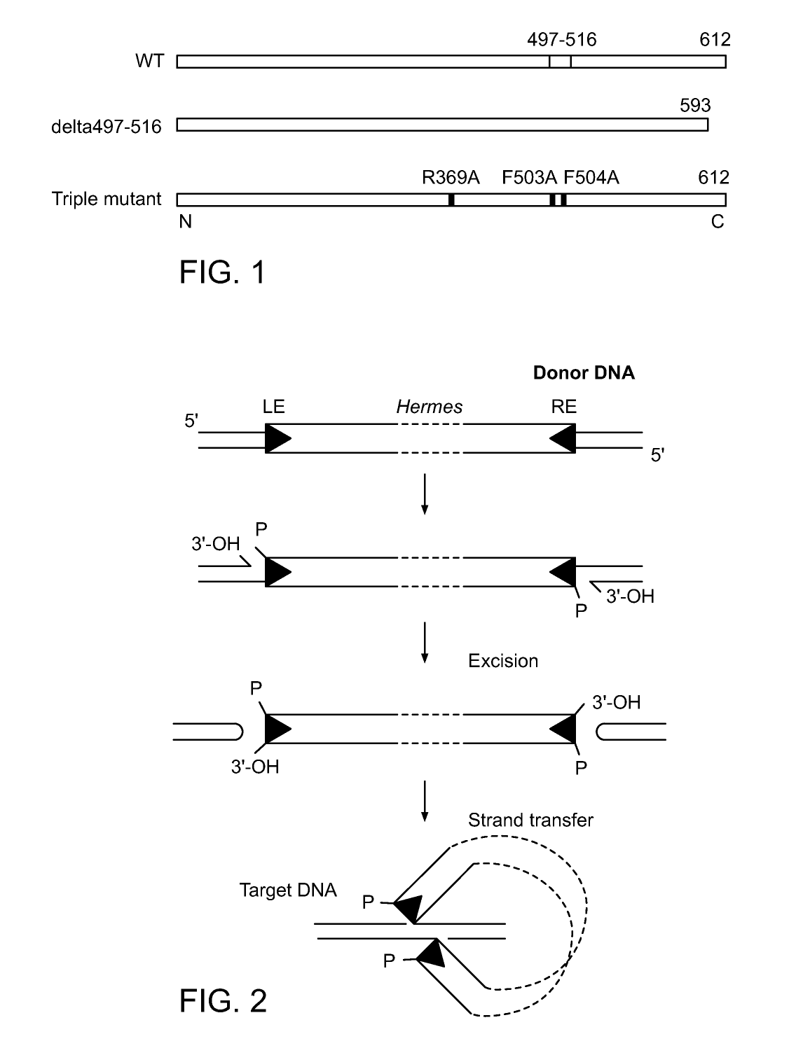

The Hermes transposase gene has since been cloned (SEQ ID NO:2) and encodes a 612 amino acid polypeptide chain (FIG. 1., SEQ ID NO:1) similar to other members of the hAT family of transposases, e.g. hobo, Ac and Tam3. The transposon is flanked by 17 bp imperfect Left (L-end) and Right (R-end) terminal inverted repeat sequences that are substrates for the transposition reaction (L-end=SEQ ID NO:3 and R-end=SEQ ID NO:4) and are similar to other members of the hAT family. Mechanisms involved in Hermes transpositions have been carefully characterized. The Hermes Protein facilitates movement of the entire Transposon element by binding initially to each of the two 17 bp terminal binding sequences. Alternatively, the left end 35mers, 30mers, 25mers, 20mers, 15mers, 10 mers, or 5mers are used as substrates. This is followed by cleavage at both ends of Donor DNA association with target DNA, then, strand transfer and the generation of 8-base-pair (bp) target-site duplications in target DNA upon transposition (5).

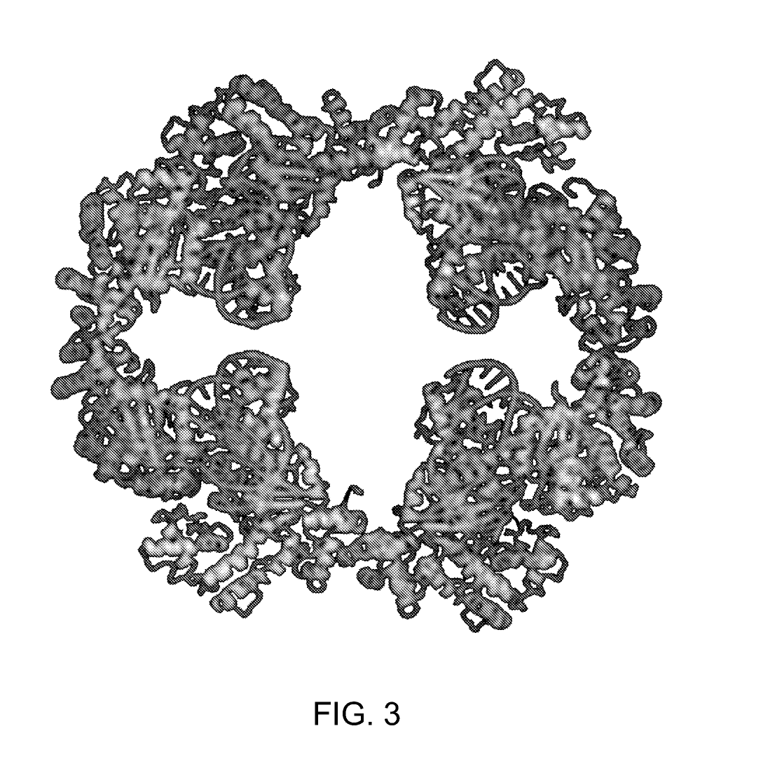

This scheme is illustrated in FIG. 2 where initial cleavage at the left ends (LE) and right ends (RE) of the Hermes element occurs one nucleotide into the flanking strand of the 5′ ends of the transposon, thereby generating a flanking 3′-OH group. Subsequent nucleophilic attack by this 3′-OH group on the opposite strand results in flanking hairpins and 3′-OH groups at either end of the transposon. These two new 3′-OH groups act as nucleophiles for a coordinated attack on target DNA, in which two insertion events, separated by 8 bp, occur on opposite strands of the Target DNA. This results in addition of lengths of the target DNA onto the transposon effectively inserting the transposon.

The full-length native Hermes transposase (Hermes; residues 1-612) was subcloned into pET-15b (Novagen) for expression in Escherichia coli as an N-terminal His-tag fusion protein and purified. The full-length Hermes transposase (residues 1-612) is soluble, but not readily amenable to crystallization for structural studies because it forms large aggregates in solution when expressed as an N-terminally histidine (His)-tagged fusion protein in E. coli. However, removal of the N-terminal 78 residues results in a version of Hermes that is readily crystallized. The structure of Hermes79-612 was solved using X-ray crystallography (6).

Size-exclusion chromatography and sedimentation equilibrium experiments revealed that Hermes forms multimers in solution and examination of the structure revealed an explanation for the multimerization of Hermes253-612 is provided by the presence of a second interface (interface 2) through which heterodimers can form heterotetramers. This interface arises by domain swapping of two helices between residues 497 and 516 that project away from each Hermes79-612 molecule.

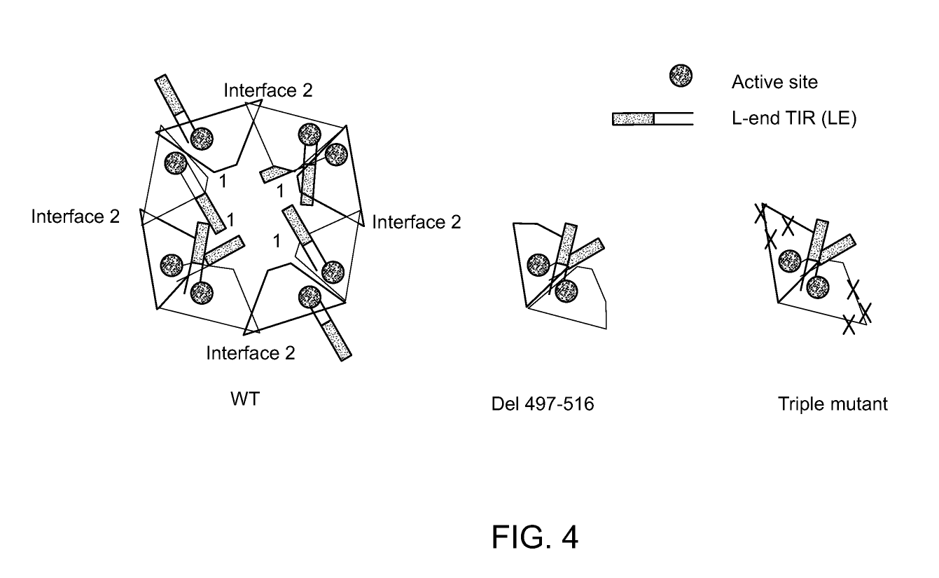

The crystal structure of Hermes79-612 as well as a more recent unpublished structure solved by Alison Hickman and others that reveals the configuration of transposon ends within this structure, see FIG. 3, which made it possible to determine residues in the protein that if mutated or deleted could alter the structure of the multimeric protein complex and its activity (7).

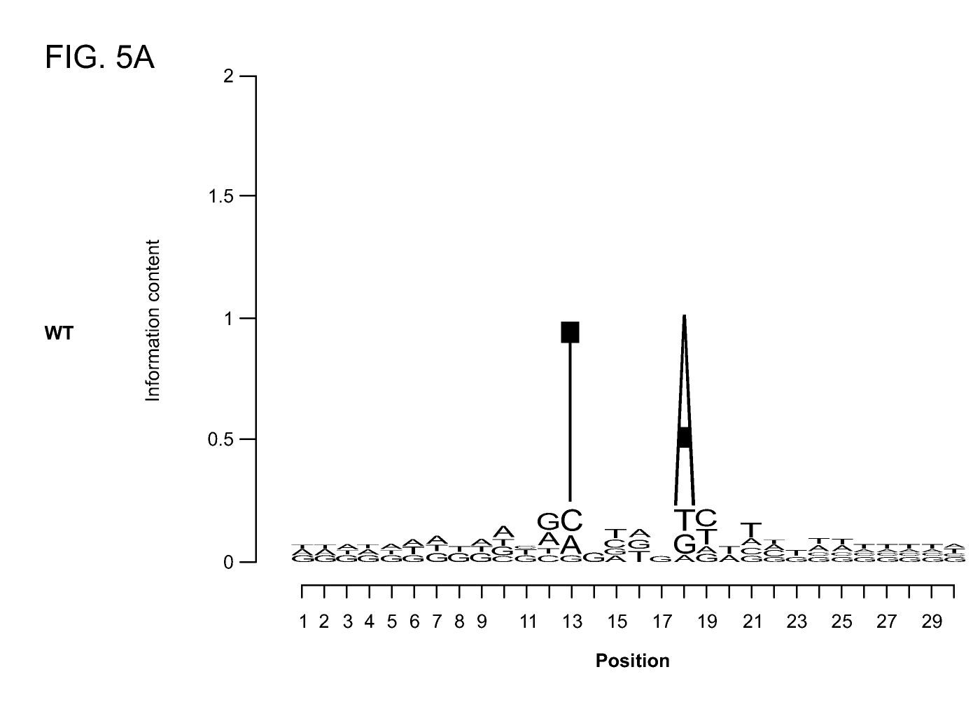

Therefore, several residues were mutated along the polypeptide chain and each mutant tested for its Transposition activity. Two mutants (FIG. 1), the “triple mutant” with a combination of three mutations of residues Arginine to Alanine at position 369, Phenylalanine to Alanine at position 503 and Phenylalanine to Alanine at position 504 in the polypeptide chain (SEQ ID NO:5 (protein), SEQ ID NO:6 (nucleic acid)), and the “delta497-516” mutant with a deletion of residues from positions 497 through to position 516 on the polypeptide chain (SEQ ID NO:7 (protein), SEQ ID NO:8 (nucleic acid)) formed dimeric complexes in solution and were more active than the native enzyme in in vitro transposition reactions at both 30° C. and at 23° C., using an dsDNA oligonucleotide with the Hermes terminal inverted repeat sequences, a target plasmid, usually pUC19 or pBR322, the purified Hermes transposase and divalent cations such as Mg²⁺ or Mn²⁺. FIG. 4 diagrammatically shows that wild type (WT) Hermes Transposase forms heterodimers which assemble into octamers through the mediation of Interface 2. Both the delta497-516 mutant and the triple mutant lack effective Interface 2s so they form only dimers in solution.

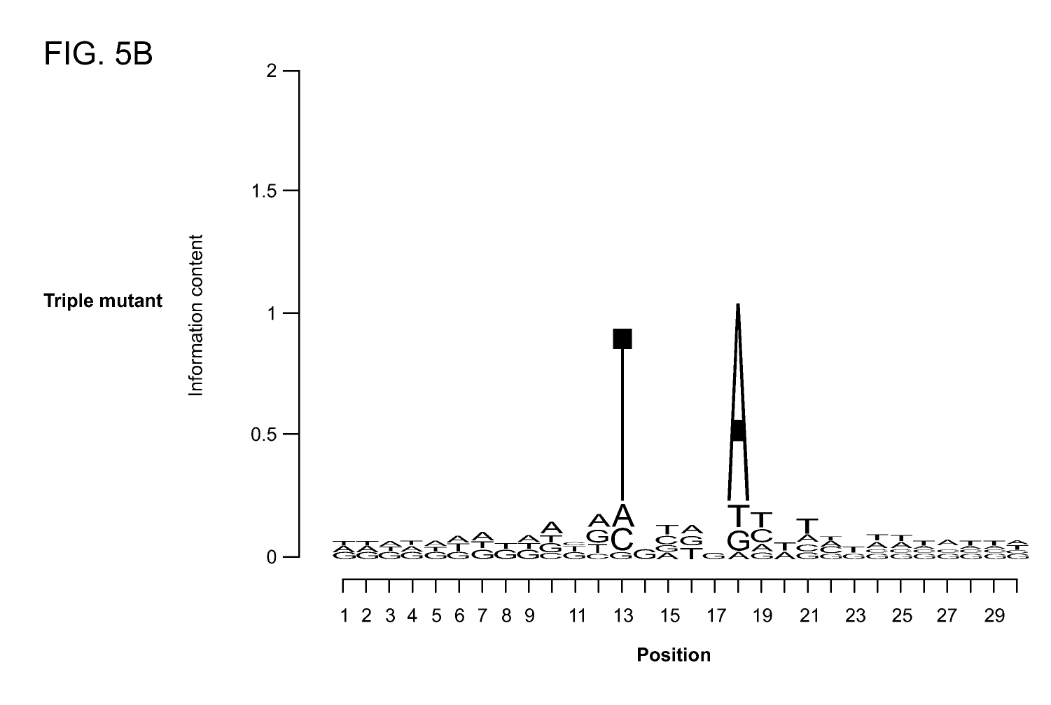

The polypeptide sequences and method of production of the “triple mutant” and the “delta497-516” mutants of Hermes Transposase for in vitro transposition and 5′ tagging of nucleic acids are disclosed herein. Methods of using the above hyperactive forms of the Hermes Transposase in generating genomic 5′ transposon tagged libraries for whole genome amplification and DNA sequencing are also disclosed. The wild type Hermes Transposase showed minimal insertional bias when a very large dataset of in vitro target sites were analyzed by using a standard method (8). Using this approach, in one example where half of a sequencing lane of an Illumina sequencing slide (Illumina, Inc., San Diego, Calif.) was used, 6.5× coverage of the yeast genome was obtained, i.e., on average, each base is contained in 6.5 reads, with only 7.02% of the genome not covered. It was confirmed that the triple mutant did not display any difference in insertional bias. FIG. 5 shows sequence logos of both the wild type (WT) and the triple mutant produced by overlaying the insertion sites of the transposases. The strong thymine and adenine consensus signals indicate essentially no difference in target site selection between the two different transposases.

### Methods of Purification of Hyperactive Hermes Transposase:

### Method 1.

The Hermes transposase (Tnsp) ORF (612 amino acids) was amplified by polymerase chain reaction (PCR) from plasmid pBCHSHH1.9v and cloned between the NcoI and PvuII sites of plasmid pBAD/Myc-HisB (Invitrogen) to generate a Hermes-Myc-His fusion construct, pLQ4. E. coli strain Top10 (Invitrogen) transformed with the Hermes-Myc-His plasmid was grown overnight with shaking at 30° C. in LB medium containing 100 mg/ml carbenicillin. The following day the overnight culture was diluted 1:100 with fresh LB+carbenicillin, and cells were then grown to an absorbance at 600 nm of 0.6 at 30° C. The culture was then shifted to 16° C. and induced with 0.1% L-arabinose for 16 h. After induction, cells were washed by centrifugation at 4° C. with TSG (20 mM Tris-HCl, pH 7.9, 500 mM NaCl, 10% v/v glycerol), and frozen in liquid nitrogen; all subsequent steps were performed at 4° C. Frozen cells were resuspended in 10 ml TSG and lysed by sonication. The cleared lysate was loaded onto a pre-equilibrated Ni²⁺ Sepharose column (Amersham) and washed with ten column volumes of TSG, six column volumes of TSG+50 mM imidazole and six column volumes of TSG+100 mM imidazole. The Henries-Myc-His fusion protein was eluted with six column volumes of TSG+200 mM imidazole, dialyzed against TSG, and stored at −80° C.

### Method 2.

Soluble Henries Transposase (both wild-type and mutants) was obtained by expression in E. coli BL21(DE3) cells which were grown at 310 K until OD600=0.6. Cells were then rapidly cooled on ice to 19° C. and protein expression was induced by addition of IPTG to a final concentration of 0.5 mM. Cells collected from an 8 liter culture were harvested 16-20 h post-induction. The pellet was resuspended in 300 mM NaCl, 12 mM phosphate pH 7.4, flash-frozen in liquid nitrogen and then stored at 193 K. Unless noted otherwise, all purification steps were performed at 4° C. After thawing, cells were lysed by sonication in the presence of 500 mM NaCl, 5 mM imidazole (Im), 25 mM Tris pH 7.5 and 2 mM β-mercaptoethanol (BME). Following centrifugation of the cell lysate at 100,000 g for 45 min, the supernatant was loaded onto a Hi-Trap metal-chelation column (Amersham Biosciences) previously equilibrated with NiSO4. The column was washed extensively with 20 mM Tris pH 7.5, 2 mM Im and 500 mM NaCl followed by the same buffer containing 22 mM Im. Hermes was eluted from the column using a gradient of 22-400 mM Im. After visualization on an SDS-PAGE gel, fractions containing Hermes 79-612 were combined and dialyzed against 20 mM Tris pH 7.5, 1 mM EDTA, 500 mM NaCl, 4 mM BME and 10% (w/v) glycerol. This was followed by dialysis against a single change of the same buffer containing 5 mM dithiothreitol (DTT) in place of BME (TSK buffer). To remove the polyhistidine tag, 10 units of thrombin (Sigma) were added per milligram of protein and incubated overnight. Thrombin was removed by passage over a 1 ml benzamidine Sepharose 4B (Pharmacia) column.

Method 3. Purification of Transposase without an Affinity Tag:

It is also possible to purify Hermes transposases in sufficient quantities by expressing a version of the protein that lacks an affinity purification tag. This was done by introducing a stop codon at the position where the sequence corresponding to the tag begins in the Hermes Transposase coding region of pLQ4 of method 1.

Protein was expressed in Top10 cells by growth at 37° C. until OD600 nm˜0.6, followed by cooling to 19° C. and then induction by addition of arabinose to a final concentration of 0.012%; cells were harvested after 16-18 hrs. Cells were lysed by sonication in Lysis Buffer (25 mM Tris pH 7.5, 0.5 M NaCl, 0.2 mM TCEP), centrifuged to remove cell debris, and the soluble material loaded onto Heparin Sepharose columns (GE Healthcare) previously equilibrated in 25 mM Tris pH 7.5, 0.1 M NaCl, 0.2 mM TCEP. After washing with the same buffer containing 0.5 M NaCl, protein was eluted using a linear gradient from 0.5 M to 1.0 M NaCl. For gel filtration, fractions containing Hermes were combined, concentrated, and loaded onto a preparative scale BioSep-SEC-S 3000 column (Phenomenex) equilibrated in 25 mM HEPES pH 7.3, 1.5 M NaCl, and 0.2 mM TCEP.

### Strand Transfer Assay:

Pre-cleaved Hermes-L end for strand-transfer reactions to measure transposition activity was made by annealing the following oligonucleotides:

(SEQ ID NO: 9)
5′-P-TCAGAGAACAACAACAAGTGGCTTATTTTGA-3′
(top)
and
(SEQ ID NO: 10)
5′-TCAAAATAAGCCACTTGTTGTTGTTCTCTG-3′
(bottom)

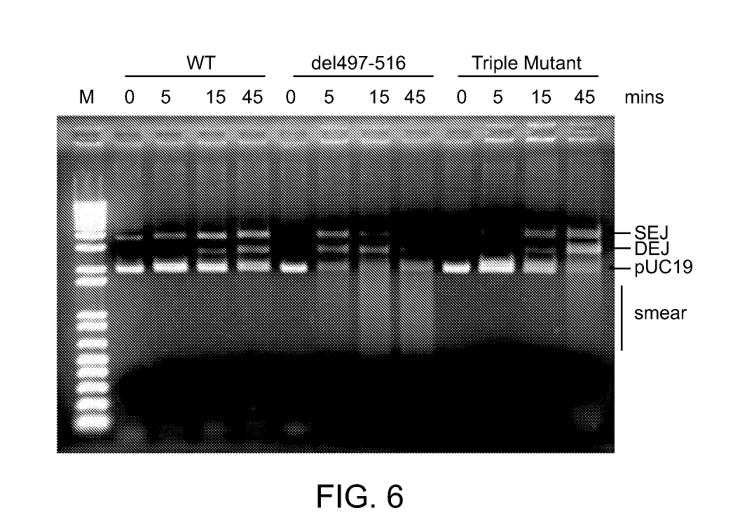

In some experiments, the oligonucleotide was radiolabeled at its 5′ end with y-P³²-dATP (to demonstrate covalent attachment to target) (9 and 10) or, as in the example shown in FIG. 6, unlabeled and used directly as a substrate at 22.9 nM or 60 nM or anywhere from 5 nM to 100 nM for strand-transfer reactions with 3.4 nM or 4 nM pUC19/pBR322 target DNAs and 5 nM to 10.7 nM of Hermes Transposase. In the experiment illustrated in FIG. 6 reactions were incubated for 0 to up to 120 minutes (times of 0, 4, 15, and 45 minutes. are shown), at 23° C. or 30° C., preferably 30° C. The reactions were stopped by addition of SDS and EDTA to a concentration of 0.5%-1% SDS and 20-25 mM EDTA, incubated at 65° C. for 20 minutes at room temperature (RT), and in some cases treated with 40 μg of proteinase K and incubated for 30 minutes at 37° C. for analysis. DNA was extracted with phenol/chloroform, precipitated with ethanol and loaded onto 1% TAE agarose gels and/or gel dried and phosphor imaged and the various end products of the reaction analyzed by their distinct electrophoretic mobility. In FIG. 6 the gels were stained with Ethidium bromide to visualize the nucleic acid bands. SEJ and DEJ represent the product of one and two insertions, respectively, per plasmid target molecule. The smear represents the products of fragmentation resulting from more than three insertions per target molecule.

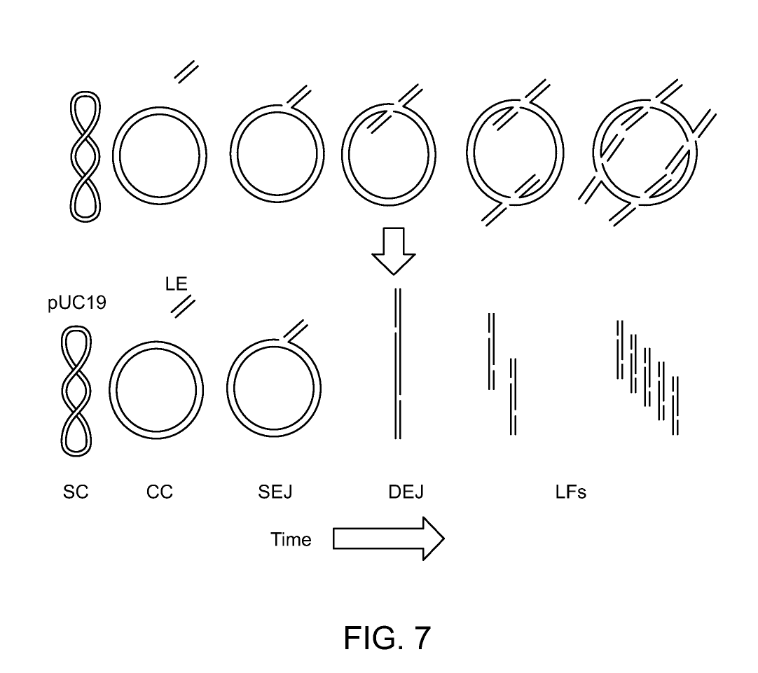

FIG. 7 diagrammatically illustrates the insertion process leading to these results. Transposon Left-end (LE) inserts into supercoiled (SC) plasmid (pUC19) DNA converting it to the nicked circular single end joined (SEJ) configuration and with an additional insertion into the linear double end joined (DEJ) form and with still more insertions into the linear fragments (LFs) that make up the smear.

The dimeric forms of Hermes Transposase are efficient in strand transfer/covalent attachment to target DNA and fragment the target DNA as the reaction proceeds as shown in FIGS. 7 and 6.

### Methods of Preparing Transposon Insertion Libraries for High-Throughput Sequencing.

### A) Strand Transfer Reaction:

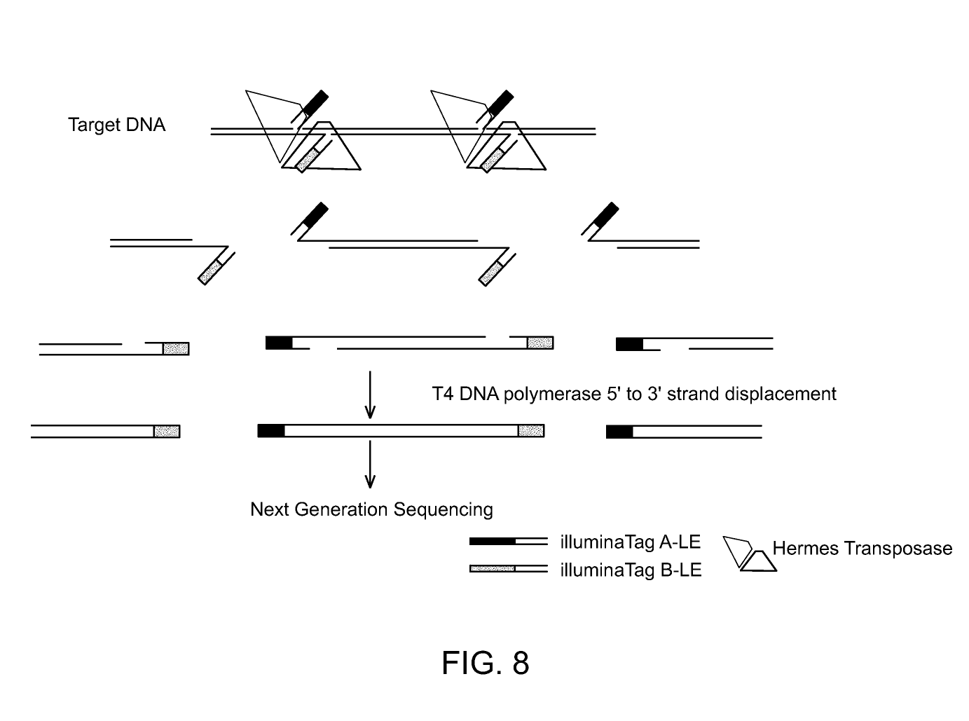

The Strand transfer reaction is diagrammatically illustrated in FIG. 8 where insertion of tagged transposon ends into target DNA results in 8-bp single stranded gaps which are filled in by strand displacing DNA polymerases such as T4 DNA polymerase. This allows Next Gen sequencing platform specific sequences to be attached to fragments of target DNA. Strand transfer reaction was carried out by mixing 285.7 nM (2 ug in 100 uL) purified Hermes transposase, 1 mM (100 pmoles in 100 uL) biotinylated double-stranded Hermes L-end oligonucleotide (LE) containing the 17 bp terminal inverted repeat, prepared by annealing oligonucleotides such as the following:

5′ Biotinylated oligo-Hermes LE Top strand, (SEQ ID No:11) 5′Biotin-ataagtagcaagtggcgcataagtatcaaaataagccaCTTGTTGTTGTTCTCTG and 5′phosphorylated oligo,-Hermes LE Bottom strand, (SEQ ID NO:12) 5′P-cCAGAGAACAACAACAAGtggcttattttgatacttatgcgccacttgctacttat (Synthesized by IDT) with the addition to 2.53 pM (2 μg in 100 μL) of proteinase K treated-phenol-chloroform purified Schizosaccharomyces pombe or Saccharomyces cerevisae genomic DNA in a buffer containing 25 mM MOPS pH 7.5, 100 mM NaCl, 10 mM MgCl2, 4% Glycerol, 2 mM DTT, 0.1 mg/mL BSA for 2-3 h at 30° C. The reaction was quenched by adding EDTA and SDS to a final concentration of 20 mM and 0.1% respectively and inactivating the enzyme at 65° C. for 20 min. Note that for SEQ ID NO:11 the uppercase nucleotides represent the 17 bp terminal inverted repeat while the lowercase nucleotides represent the biotin sequencing priming region. For SEQ ID NO:12 the uppercase nucleotides represent the 17 bp terminal inverted repeat while the lowercase nucleotides represent the sequencing priming region.

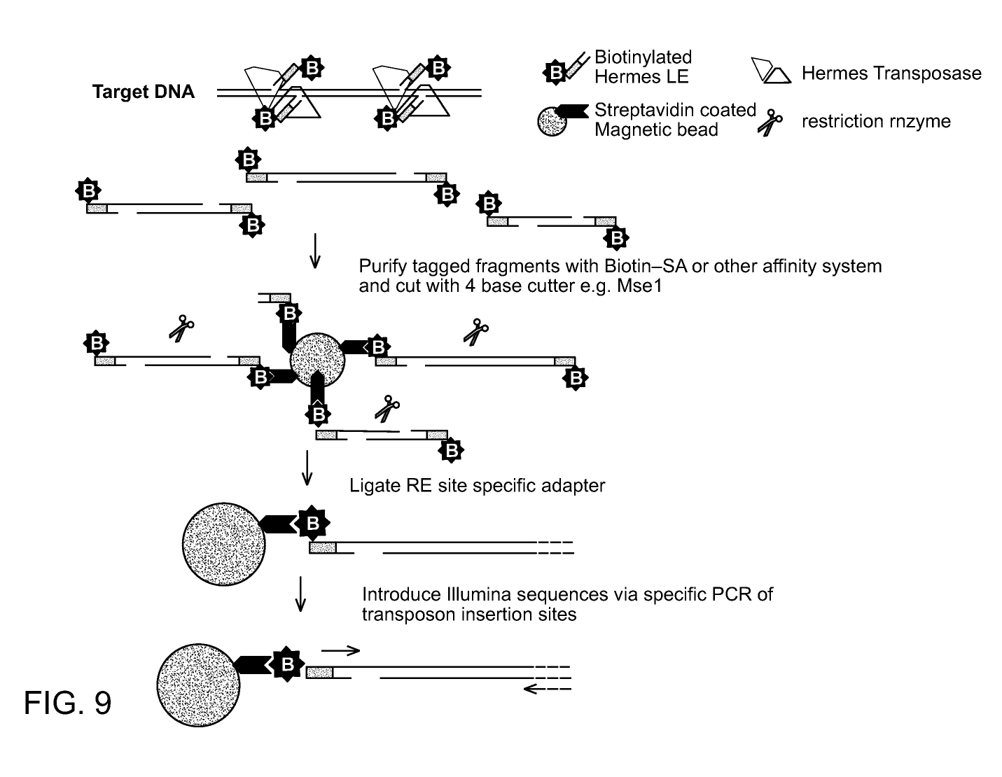

At this stage as shown in FIG. 9, the 3′ end of the top strand of the biotinylated double stranded transposon LE is covalently attached to the 5′ of the target DNA fragment on two ends and fragmentation of the target DNA has occurred along its length. Streptavidin (SA) beads or other affinity systems can be used to purify the tagged fragments. After which the fragments can be cut with a four base cutter such as Msel. There are several well-known methods for modifying these fragments so that they are prepared as suitable templates for DNA sequencing. For example, specific Next gen sequencing tags such as Illumina sequences can be introduced via specific PCR of the insertion sites. Well-known methods are used to fill in the 8 bp gaps in the fragments.

### B) Methods of Preparing the Transposase Mediated Fragmented and 5′ Tagged DNA for Sequencing:

The fragments can, at this stage, be subjected to an extension and strand displacement reaction using DNA polymerase. Arbitrary tags or specific Next gen sequencing platform specific tags (e.g. SEQ ID NOs:17-20) can be added onto the target DNA fragments by this method (see FIG. 9). This method also requires designing primers complementary to the transposon ends in such way that a “suppression PCR” can produce the 5′ (Arbitrary tag A-(LE)) and 3′ (Arbitrary tag B-LE)) Next Gen sequencing tags (as in the Nextera kit, Illumina) on either end of each of the fragments.

Hermes L-end oligo (tag A-LE) with Illumina/arbitrary tag A sequencing priming region, 4 bp barcode and a 30 bp Hermes Transposon end is prepared by annealing:

tagA-LE top strand (SEQ ID NO: 17):
5′ Biotin
AATGATACGGCGACCACCGAGATCTacactctttccctacacgacgctct
tccgatctGCGTtcaaaataagccacTTGTTGTTGTTCTCTG
and
a tagA-LE bottom strand (SEQ ID NO: 18):
5′Phospho
cCAGAGAACAACAACAAgtggcttattttgaACGCagatcggaagagcgt
cgtgtagggaaagagtgtAGATCTCGGTGGTCGCCGTATCATT.

For SEQ ID NO:17 the Illumina/arbitrary tag A is shown in uppercase while the sequencing priming region is shown in lower case with the 4 bp barcode in uppercase followed by a 30 bp Hermes Transposon end with the minimal 17 bp end shown in lower and uppercase. For SEQ ID NO:18 the 30 bp Hermes Transposon end with the minimal 17 bp end is shown in uppercase and lowercase with the 4 bp barcode in uppercase followed by the sequencing priming region in lowercase and the-Illumina/arbitrary tag A in uppercase.

A Henries L-end oligo (tagB-LE) with Illumina/arbitrary tag A sequencing priming region, 4 bp barcode and 30 bp Henries Transposon end is prepared by annealing

tagB-LE top strand (SEQ ID NO: 19):
CAAGCAGAAGACGGCATACGAGCTCacactctttccctacacgacgctct
tccgatctGCGTtcaaaataagccacTTGTTGTTGTTCTCTG
and
tag B-LE bottom strand (SEQ ID NO: 20):
cCAGAGAACAACAACAAgtggcttattttgaACGCagatcggaagagcgt
cgtgtagggaaagagtgtGAGCTCGTATGCCGTCTTCTGCTTG.

For SEQ ID NO:19 the Illumina/arbitrary tag B is shown in uppercase, the sequencing priming region is shown in lower case followed by a 4 bp barcode in uppercase and a 30 bp Hermes Transposon end with the minimal 17 bp end shown in lowercase and uppercase. For SEQ ID NO:20 the 30 bp Henries Transposon end with the minimal 17 bp end is shown in lowercase and uppercase followed by a 4 bp barcode in uppercase and a sequencing priming region and Illumina/arbitrary tag B in uppercase.

Arbitrary tags or specific Next gen sequencing platform specific tags can also be added onto the target DNA fragments by a modified method that does not need “suppression PCR” but provides a second distinct priming site using any “4-bp cutter”-restriction enzyme and a linker ligation mediated PCR approach.

In this method as shown in FIG. 9, the fragments attached to the biotinylated transferred strand are bound to magnetic Streptavidin coupled Dynal beads (Invitrogen) in binding and washing buffer (B & W buffer: 100 mM Tris.HCl, pH 8.0, 1 mM EDTA, and 1M NaCl). The B & W buffer is removed after magnetic separation and the beads resuspended in a digestion mix that contains a restriction enzyme, e.g., MseI that cuts at TTAA (NEB). Basically, a variety of affinity purification systems are adaptable to this and related methods. Various types of ligand-binding molecule systems are usable as well. Most often the small ligand is attached to the transposon and the binding molecule (receptor) is attached to a solid phase. In the illustrated examples the solid phase is composed of magnetic beads, but the solid phase can also be beads or solids in a chromatographic column or solid surfaces on a chip, etc. Biotin-Streptavidin and polyhistidine (more than six histidine residues)-nickel/cobalt binding moieties are illustrated. Lectin-sugar and hapten-antibody systems as well as other affinity systems can be used.

The bound DNA is digested at 37° C. overnight. The beads are washed and Msel-specific linkers (obtained by annealing Linker/adapter Top strand (SEQ ID NO:13) and Linker/adapter bottom strand (SEQ ID NO:14) are ligated to the Msel-digested ends of the Henries L-end attached DNA. The beads are washed to remove non-ligated linkers. The DNA bound to the beads are used as a template for the PCR amplification of the Henries L-end insertion site junctions using the 5′ transposon end specific primer, that has i) 5′ Illumina tag sequence fused to ii) an Illumina proprietary sequence (sequencing primer), 4-by barcode and the Hermes L-end complementary sequence (SEQ ID NO:15) and the 3′ linker/adapter specific primer, that has the 3′ Illumina tag (SEQ ID NO:16). The PCR mix is separated from the Dynal beads, concentrated, the amplicons size-selected on an agarose gel and purified by gel extraction. Massively parallel sequencing is then carried out on the illumina Hi-Seq HTS platform.

The linker/adapter Top strand is SEQ ID NO: 13:
TAGTCCCTTAAGCGGAGCCCTATAGTGAGTCGTATTAC.
The linker/adapter bottom strand is SEQ ID NO: 14:
GTAATACGACTCACTATAGGGCTCCGCTTAAGGGAC.
The 5′ Transposon end specific primer is
SEQ ID NO: 15:
AATGATACGGCGACCACCGAGATCTacactctttccctacacgacgctct
tccgatctGCGTcgcataagtatcaaaataagccac.
The 3′ linker/adapter specific primer is
SEQ ID NO: 16:
CAAGCAGAAGACGGCATACGAGCTCttccgatctgtaatacgactcacta
tagggc.

For SEQ ID NO:15 the Illumina tag A and the 4 bp barcode are in uppercase while the sequencing priming region and inverted repeat are in lowercase. For SEQ ID NO:15 the Illumina tag B is in uppercase while the linker adapter PCR priming region is in lower case.

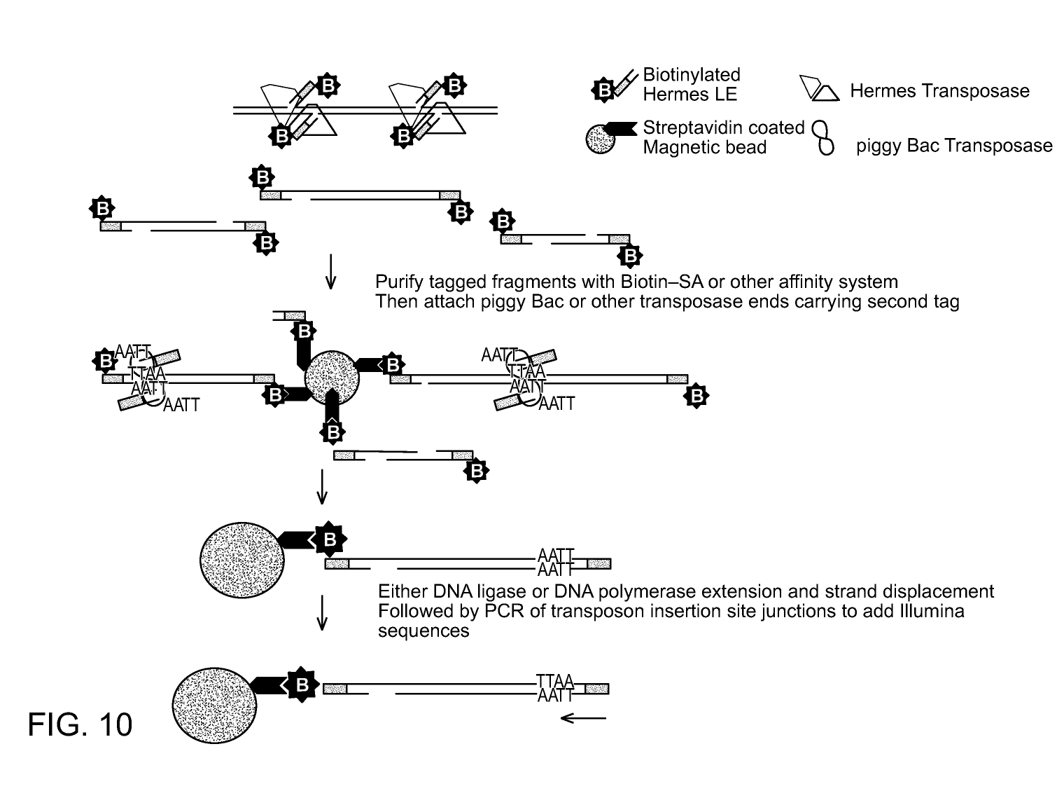

In another variation of the above embodiment (shown in FIG. 10), after tagging the 5′ ends of the target genomic DNA by strand transfer with biotinylated Henries transposon end, instead of restriction digestion and linker ligation, a second transposase is used to provide the second tag (with a priming site distinct from the priming site provided by the Henries transposon end) after capturing the fragments on magnetic beads. The second transposon may preferably be the piggy Bac transposase that is disclosed in and covered by Published Patent Applications US 2010/0287633, US 2010/0154070, and US 2007/0204356 (which are incorporated herein by reference to the extent allowed by applicable statute or regulation). However, any other transposase that has target DNA recognition characteristics distinct from Hermes such as SPIN, AeBuster, or even Mu and Tn5 (Nextera) can be used. This step is followed by DNA polymerase mediated extension and strand displacement using T4 DNA polymerase or DNA ligation using T4 ligase followed by PCR using primers carrying Next Gen sequencing primers.

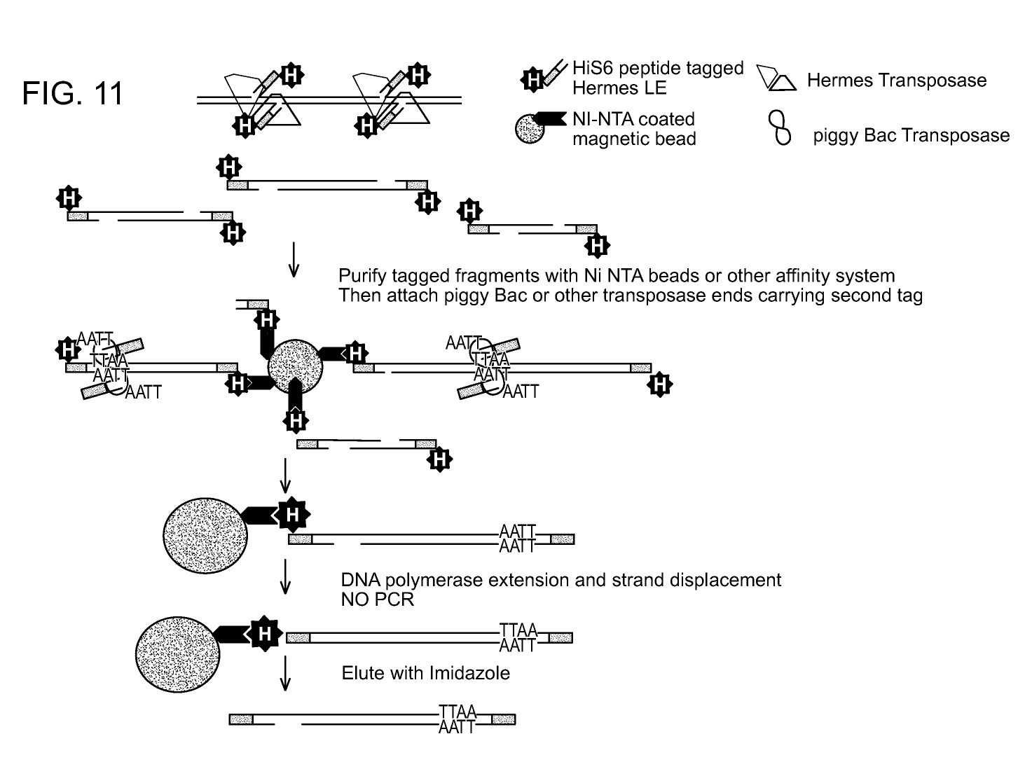

Yet another variation (shown in FIG. 11) of the above methods involves using an affinity tag, for example HIS6 (polyhistidine) peptide, covalently linked to the top strand of the transferred transposon end so that PCR amplified DNA is to be avoided prior to sequencing. In this method the DNA fragments with 8 bp single strand gaps after being immobilized on an Ni-NTA coated magnetic bead can be filled by extension and strand displacement using T4 DNA polymerase and eluted from the column using imidazole.

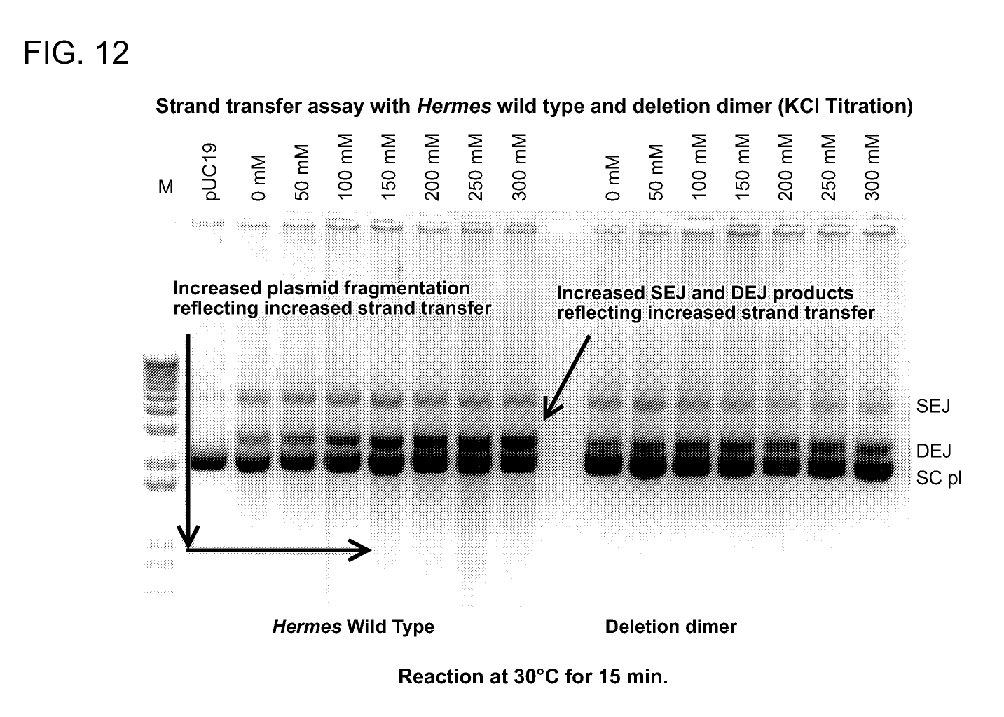

Also described herein are Hermes transposases and reaction conditions (25 mM HEPES pH 7.5, 5 mM MgCl2, 5 mM NaCl, 100-300 mM KCl, 10 mM DTT, 0.1 mg/ml BSA, 250 nanogram pUC19, 100 ng L end Hermes, 60 ng Hermes) that result in increased strand transfer in vitro, thereby increasing the efficiency of nucleic acid modification. Also described herein is the use of the wild type version of Hermes that may also be modified by mutation (e.g., C519S) to prevent aggregation at particular KCL concentrations (e.g., 100-300 mM KCL). FIG. 12 shows a photograph of an agarose gel showing the results of a strand transfer assay with Hermes wild type and a deletion dimer (KCL titration).

The materials and methods for FIG. 12 are as follows: 100 microliter reactions containing 25 mM HEPES pH 7.5, 5 mM MgCl2, 5 mM NaCl, 0-300 mM KCl as indicated, 10 mM DTT, 0.1 mg/ml BSA, 250 nanogram pUC19, 100 ng L end Hermes, 60 ng Hermes25 mM HEPES pH 7.5, 5 mM MgCl2, 5 mM NaCl, 100-300 mM KCl, 10 mM DTT, 0.1 mg/ml BSA, 250 nanogram pUC19, 100 ng L end Hermes, 60 ng Henries were incubated at 10 minutes at 30° C. The reactions were stopped by addition of 10 microliter SDS/EDTA to bring to a final concentration of 0.25% SDS and 25 mM EDTA and further incubated at 65° C. for 10 minutes. Reaction mixtures were phenol extracted, DNA ethanol precipitated and resuspended in 20 microliter H20 and displayed on a 1.5% TAE-agarose gel and run in 0.5×TAE. Gel was stained with ethidium bromide and photographed on a UV-transilluminator.

The sequence of Hermes wild type from PLQ4-Hickman (Q2E because of cloning) with added TGA STOP codon after C-terminal I612 (Hermes1-612 wildtype; SEQ ID NO: 21) is as follows:

MEKMDNLEVKAKINQGLYKITPRHKGTSFIWNVLADIQKEDDTLVEGWVF
CRKCEKVLKYTTRQTSNLCRHKCCASLKQSRELKTVSADCKKEAIEKCAQ
WVVRDCRPFSAVSGSGFIDMIKFFIKVGAEYGEHVNVEELLPSPITLSRK
VTSDAKEKKALISREIKSAVEKDGASATIDLWTDNYIKRNFLGVTLHYHE
NNELRDLILGLKSLDFERSTAENIYKKLKAIFSQFNVEDLSSIKFVTDRG
ANVVKSLANNIRINCSSHLLSNVLENSFEETPELNMPILACKNIVKYFKK
ANLQHRLRSSLKSECPTRWNSTYTMLRSILDNWESVIQILSEAGETQRIV
HINKSIIQTMVNILDGFERIFKELQTCSSPSLCFVVPSILKVKEICSPDV
GDVADIAKLKVNIIKNVRIIWEENLSIWHYTAFFFYPPALHMQQEKVAQI
KEFCLSKMEDLELINRMSSFNELSATQLNQSDSNSHNSIDLTSHSKDIST
TSFFFPQLTQNNSREPPVCPSDEFEFYRKEIVILSEDFKVMEWWNLNSKK
YPKLSKLALSLLSIPASSAASERTFSLAGNIITEKRNRIGQQTVDSLLFL
NSFYKNFCKLDI*

The sequence of Hermes C519S from pLQ4-Hickman Hermes Q2E; C519S, with STOP added after C-terminal I612 (Hermes1-612 C519S; SEQ ID NO: 22) is as follows:

MEKMDNLEVKAKINQGLYKITPRHKGTSFIWNVLADIQKEDDTLVEGWVF
CRKCEKVLKYTTRQTSNLCRHKCCASLKQSRELKTVSADCKKEAIEKCAQ
WVVRDCRPFSAVSGSGFIDMIKFFIKVGAEYGEHVNVEELLPSPITLSRK
VTSDAKEKKALISREIKSAVEKDGASATIDLWTDNYIKRNFLGVTLHYHE
NNELRDLILGLKSLDFERSTAENIYKKLKAIFSQFNVEDLSSIKFVTDRG
ANVVKSLANNIRINCSSHLLSNVLENSFEETPELNMPILACKNIVKYFKK
ANLQHRLRSSLKSECPTRWNSTYTMLRSILDNWESVIQILSEAGETQRIV
HINKSIIQTMVNILDGFERIFKELQTCSSPSLCFVVPSILKVKEICSPDV
GDVADIAKLKVNIIKNVRIIWEENLSIWHYTAFFFYPPALHMQQEKVAQI
KEFCLSKMEDLELINRMSSFNELSATQLNQSDSNSHNSIDLTSHSKDIST
TSFFFPQLTQNNSREPPVSPSDEFEFYRKEIVILSEDFKVMEWWNLNSKK
YPKLSKLALSLLSIPASSAASERTFSLAGNIITEKRNRIGQQTVDSLLFL
NSFYKNFCKLDI*

The sequence of plasmid pLQ4-Hickman Hermes Q2E/STOP after Hermes I612 (Hermes ORF in UC) (pLQ4 Hermes1-612 wildtype “613” STOP; SEQ ID NO: 23) is as follows:

aagaaaccaattgtccatattgcatcagacattgccgtcactgcgtcttt
tactggctcttctcgctaaccaaaccggtaaccccgcttattaaaagcat
tctgtaacaaagcgggaccaaagccatgacaaaaacgcgtaacaaaagtg
tctataatcacggcagaaaagtccacattgattatttgcacggcgtcaca
ctttgctatgccatagcatttttatccataagattagcggatcctacctg
acgctttttatcgcaactctctactgtttctccatacccgttttttgggc
taacaggaggaattaaccATGGAGAAAATGGACAATTTGGAAGTGAAAGC
AAAAATCAACCAAGGATTATATAAAATTACTCCGCGACATAAAGGAACAA
GTTTTATTTGGAACGTTTTAGCGGATATACAGAAAGAAGACGATACATTG
GTGGAAGGGTGGGTGTTTTGCCGAAAATGCGAAAAAGTTTTAAAATACAC
AACTAGGCAGACATCAAACTTATGTCGTCATAAATGCTGTGCCTCTCTAA
AGCAATCCCGAGAATTAAAAACTGTTTCAGCTGATTGCAAAAAGGAAGCA
ATTGAAAAATGTGCACAATGGGTGGTACGAGATTGTCGGCCTTTTTCGGC
CGTCTCTGGATCCGGCTTTATCGATATGATAAAATTTTTTATTAAAGTTG
GAGCCGAATATGGTGAACATGTCAACGTTGAGGAATTGTTACCAAGTCCA
ATAACGCTATCGAGAAAGGTAACTTCGGATGCAAAAGAAAAAAAAGCTCT
GATTAGTCGAGAAATTAAGTCTGCTGTAGAGAAAGATGGTGCATCAGCAA
CGATAGATTTGTGGACCGATAATTATATAAAACGGAATTTTTTGGGAGTA
ACGTTACACTACCATGAAAACAATGAACTGCGAGATCTAATTTTAGGTTT
AAAGTCCTTAGATTTTGAAAGATCCACAGCAGAAAATATTTATAAGAAGC
TTAAAGCCATTTTTTCACAATTCAACGTCGAAGACTTGAGTAGTATAAAA
TTTGTGACAGATAGAGGAGCCAATGTCGTAAAATCATTGGCAAATAATAT
CAGAATTAACTGCAGCAGCCATTTGCTTTCAAACGTGTTGGAAAATTCAT
TTGAGGAGACACCTGAACTCAATATGCCTATTCTTGCTTGCAAAAATATT
GTAAAATATTTCAAGAAAGCCAATCTGCAGCACAGACTTCGAAGTTCTTT
AAAAAGTGAGTGCCCTACACGGTGGAATTCCACATACACGATGCTTCGAT
CTATTCTCGACAACTGGGAAAGCGTGATTCAAATATTAAGTGAGGCGGGA
GAGACACAGAGAATTGTTCATATAAATAAGTCGATAATTCAAACAATGGT
CAACATCCTCGATGGGTTTGAAAGAATTTTTAAAGAATTACAAACATGCA
GTTCACCATCTCTGTGTTTTGTTGTGCCTTCCATTTTAAAAGTAAAAGAA
ATATGTTCACCTGACGTTGGCGACGTTGCAGATATAGCAAAATTGAAAGT
GAACATTATAAAAAATGTAAGAATAATATGGGAAGAAAATTTAAGCATAT
GGCACTACACAGCATTTTTTTTCTATCCGCCCGCCTTGCATATGCAACAA
GAGAAAGTGGCACAAATTAAAGAATTTTGCTTATCCAAAATGGAAGATTT
GGAATTAATAAACCGCATGAGTTCCTTTAACGAATTATCCGCAACTCAGC
TTAACCAGTCGGACTCCAATAGCCACAACAGTATAGATTTAACATCCCAT
TCAAAAGACATTTCAACGACAAGTTTCTTTTTCCCGCAATTAACTCAGAA
CAATAGTCGTGAGCCACCAGTGTGTCCAAGCGATGAATTTGAATTTTATC
GTAAAGAAATAGTTATTTTAAGCGAAGATTTTAAAGTTATGGAATGGTGG
AATCTTAATTCAAAAAAGTATCCTAAACTATCTAAACTGGCTTTGTCGTT
ATTATCAATACCTGCAAGTAGCGCTGCATCGGAAAGGACATTTTCCCTAG
CTGGAAATATAATAACTGAAAAGAGAAACAGGATTGGGCAACAAACTGTC
GACAGCTTGTTATTTTTAAATTCCTTTTACAAAAATTTTTGTAAATTAGA
TATATGActggtaccatatgggaattcgaagcttgggcccgaacaaaaac
tcatctcagaagaggatctgaatagcgccgtcgaccatcatcatcatcat
cattgagtttaaacggtctccagcttggctgttttggcggatgagagaag
attttcagcctgatacagattaaatcagaacgcagaagcggtctgataaa
acagaatttgcctggcggcagtagcgcggtggtcccacctgaccccatgc
cgaactcagaagtgaaacgccgtagcgccgatggtagtgtggggtctccc
catgcgagagtagggaactgccaggcatcaaataaaacgaaaggctcagt
cgaaagactgggcctttcgttttatctgttgtttgtcggtgaacgctctc
ctgagtaggacaaatccgccgggagcggatttgaacgttgcgaagcaacg
gcccggagggtggcgggcaggacgcccgccataaactgccaggcatcaaa
ttaagcagaaggccatcctgacggatggcctttttgcgtttctacaaact
cttttgtttatttttctaaatacattcaaatatgtatccgctcatgagac
aataaccctgataaatgcttcaataatattgaaaaaggaagagtatgagt
attcaacatttccgtgtcgcccttattcccttttttgcggcattttgcct
tcctgtttttgctcacccagaaacgctggtgaaagtaaaagatgctgaag
atcagttgggtgcacgagtgggttacatcgaactggatctcaacagcggt
aagatccttgagagttttcgccccgaagaacgttttccaatgatgagcac
ttttaaagttctgctatgtggcgcggtattatcccgtgttgacgccgggc
aagagcaactcggtcgccgcatacactattctcagaatgacttggttgag
tactcaccagtcacagaaaagcatcttacggatggcatgacagtaagaga
attatgcagtgctgccataaccatgagtgataacactgcggccaacttac
actgacaacgatcggaggaccgaaggagctaaccgcttttttgcacaaca
tgggggatcatgtaactcgccttgatcgttgggaaccggagctgaatgaa
gccataccaaacgacgagcgtgacaccacgatgcctgtagcaatggcaac
aacgttgcgcaaactattaactggcgaactacttactctagcttcccggc
aacaattaatagactggatggaggcggataaagttgcaggaccacttctg
cgctcggcccttccggctggctggtttattgctgataaatctggagccgg
tgagcgtgggtctcgcggtatcattgcagcactggggccagatggtaagc
cctcccgtatcgtagttatctacacgacggggagtcaggcaactatggat
gaacgaaatagacagatcgctgagataggtgcctcactgattaagcattg
gtaactgtcagaccaagtttactcatatatactttagattgatttaaaac
ttcatttttaatttaaaaggatctaggtgaagatcctttagataatctca
tgaccaaaatcccttaacgtgagttttcgttccactgagcgtcagacccc
gtagaaaagatcaaaggatcttcttgagatcctttttttctgcgcgtaat
ctgctgcttgcaaacaaaaaaaccaccgctaccagcggtggtttgtttgc
cggatcaagagctaccaactctttttccgaaggtaactggcttcagcaga
gcgcagataccaaatactgtccttctagtgtagccgtagttaggccacca
cttcaagaactctgtagcaccgcctacatacctcgctctgctaatcctgt
taccagtggctgctgccagtggcgataagtcgtgtcttaccgggttggac
tcaagacgatagttaccggataaggcgcagcggtcgggctgaacgggggg
ttcgtgcacacagcccagcttggagcgaacgacctacaccgaactgagat
acctacagcgtgagctatgagaaagcgccacgcttcccgaagggagaaag
gcggacaggtatccggtaagcggcagggtcggaacaggagagcgcacgag
ggagcttccagggggaaacgcctggtatctttatagtcctgtcgggtttc
gccacctctgacttgagcgtcgatttttgtgatgctcgtcaggggggcgg
agcctatggaaaaacgccagcaacgcggcctttttacggttcctggcctt
ttgctggccttttgctcacatgttctttcctgcgttatcccctgattctg
tggataaccgtattaccgcctttgagtgagctgataccgctcgccgcagc
cgaacgaccgagcgcagcgagtcagtgagcgaggaagcggaagagcgcct
gatgcggtattttctccttacgcatctgtgcggtatttcacaccgcatat
ggtgcactctcagtacaatctgctctgatgccgcatagttaagccagtat
acactccgctatcgctacgtgactgggtcatggctgcgccccgacacccg
ccaacacccgctgacgcgccctgacgggcttgtctgctcccggcatccgc
ttacagacaagctgtgaccgtctccgggagctgcatgtgtcagaggtttt
caccgtcatcaccgaaacgcgcgaggcagcagatcaattcgcgcgcgaag
gcgaagcggcatgcataatgtgcctgtcaaatggacgaagcagggattct
gcaaaccctatgctactccgtcaagccgtcaattgtctgattcgttacca
attatgacaacttgacggctacatcattcactattcttcacaaccggcac
ggaactcgctcgggctggccccggtgcattttttaaatacccgcgagaaa
tagagttgatcgtcaaaaccaacattgcgaccgacggtggcgataggcat
ccgggtggtgctcaaaagcagcttcgcctggctgatacgttggtcctcgc
gccagcttaagacgctaatccctaactgctggcggaaaagatgtgacaga
cgcgacggcgacaagcaaacatgctgtgcgacgctggcgatatcaaaatt
gctgtctgccaggtgatcgctgatgtactgacaagcctcgcgtacccgat
tatccatcggtggatggagcgactcgttaatcgcttccatgcgccgcagt
aacaattgctcaagcagatttatcgccagcagctccgaatagcgcccttc
cccttgcccggcgttaatgatttgcccaaacaggtcgctgaaatgcggct
ggtgcgcttcatccgggcgaaagaaccccgtattggcaaatattgacggc
cagttaagccattcatgccagtaggcgcgcggacgaaagtaaacccactg
gtgataccattcgcgagcctccggatgacgaccgtagtgatgaatctctc
ctggcgggaacagcaaaatatcacccggtcggcaaacaaattctcgtccc
tgatttttcaccaccccctgaccgcgaatggtgagattgagaatataacc
tttcattcccagcggtcggtcgataaaaaaatcgagataaccgttggcct
caatcggcgttaaacccgccaccagatgggcattaaacgagtatcccggc
agcaggggatcattttgcgcttcagccatacttttcatactcccgccatt
cagag

The sequence of plasmid pLQ4-Hickman Q2E; C519S; STOP after Hermes I612 (Hermes ORF in UC) (pLQ4 Hermes1-612 C519S “613” STOP; SEQ ID NO: 24) is as follows:

aagaaaccaattgtccatattgcatcagacattgccgtcactgcgtcttt
tactggctcttctcgctaaccaaaccggtaaccccgcttattaaaagcat
tctgtaacaaagcgggaccaaagccatgacaaaaacgcgtaacaaaagtg
tctataatcacggcagaaaagtccacattgattatttgcacggcgtcaca
ctttgctatgccatagcatttttatccataagattagcggatcctacctg
acgctttttatcgcaactctctactgtttctccatacccgttttttgggc
taacaggaggaattaaccATGGAGAAAATGGACAATTTGGAAGTGAAAGC
AAAAATCAACCAAGGATTATATAAAATTACTCCGCGACATAAAGGAACAA
GTTTTATTTGGAACGTTTTAGCGGATATACAGAAAGAAGACGATACATTG
GTGGAAGGGTGGGTGTTTTGCCGAAAATGCGAAAAAGTTTTAAAATACAC
AACTAGGCAGACATCAAACTTATGTCGTCATAAATGCTGTGCCTCTCTAA
AGCAATCCCGAGAATTAAAAACTGTTTCAGCTGATTGCAAAAAGGAAGCA
ATTGAAAAATGTGCACAATGGGTGGTACGAGATTGTCGGCCTTTTTCGGC
CGTCTCTGGATCCGGCTTTATCGATATGATAAAATTTTTTATTAAAGTTG
GAGCCGAATATGGTGAACATGTCAACGTTGAGGAATTGTTACCAAGTCCA
ATAACGCTATCGAGAAAGGTAACTTCGGATGCAAAAGAAAAAAAAGCTCT
GATTAGTCGAGAAATTAAGTCTGCTGTAGAGAAAGATGGTGCATCAGCAA
CGATAGATTTGTGGACCGATAATTATATAAAACGGAATTTTTTGGGAGTA
ACGTTACACTACCATGAAAACAATGAACTGCGAGATCTAATTTTAGGTTT
AAAGTCCTTAGATTTTGAAAGATCCACAGCAGAAAATATTTATAAGAAGC
TTAAAGCCATTTTTTCACAATTCAACGTCGAAGACTTGAGTAGTATAAAA
TTTGTGACAGATAGAGGAGCCAATGTCGTAAAATCATTGGCAAATAATAT
CAGAATTAACTGCAGCAGCCATTTGCTTTCAAACGTGTTGGAAAATTCAT
TTGAGGAGACACCTGAACTCAATATGCCTATTCTTGCTTGCAAAAATATT
GTAAAATATTTCAAGAAAGCCAATCTGCAGCACAGACTTCGAAGTTCTTT
AAAAAGTGAGTGCCCTACACGGTGGAATTCCACATACACGATGCTTCGAT
CTATTCTCGACAACTGGGAAAGCGTGATTCAAATATTAAGTGAGGCGGGA
GAGACACAGAGAATTGTTCATATAAATAAGTCGATAATTCAAACAATGGT
CAACATCCTCGATGGGTTTGAAAGAATTTTTAAAGAATTACAAACATGCA
GTTCACCATCTCTGTGTTTTGTTGTGCCTTCCATTTTAAAAGTAAAAGAA
ATATGTTCACCTGACGTTGGCGACGTTGCAGATATAGCAAAATTGAAAGT
GAACATTATAAAAAATGTAAGAATAATATGGGAAGAAAATTTAAGCATAT
GGCACTACACAGCATTTTTTTTCTATCCGCCCGCCTTGCATATGCAACAA
GAGAAAGTGGCACAAATTAAAGAATTTTGCTTATCCAAAATGGAAGATTT
GGAATTAATAAACCGCATGAGTTCCTTTAACGAATTATCCGCAACTCAGC
TTAACCAGTCGGACTCCAATAGCCACAACAGTATAGATTTAACATCCCAT
TCAAAAGACATTTCAACGACAAGTTTCTTTTTCCCGCAATTAACTCAGAA
CAATAGTCGTGAGCCACCAGTGAGTCCAAGCGATGAATTTGAATTTTATC
GTAAAGAAATAGTTATTTTAAGCGAAGATTTTAAAGTTATGGAATGGTGG
AATCTTAATTCAAAAAAGTATCCTAAACTATCTAAACTGGCTTTGTCGTT
ATTATCAATACCTGCAAGTAGCGCTGCATCGGAAAGGACATTTTCCCTAG
CTGGAAATATAATAACTGAAAAGAGAAACAGGATTGGGCAACAAACTGTC
GACAGCTTGTTATTTTTAAATTCCTTTTACAAAAATTTTTGTAAATTAGA
TATATGActggtaccatatgggaattcgaagcttgggcccgaacaaaaac
tcatctcagaagaggatctgaatagcgccgtcgaccatcatcatcatcat
cattgagtttaaacggtctccagcttggctgttttggcggatgagagaag
attttcagcctgatacagattaaatcagaacgcagaagcggtctgataaa
acagaatttgcctggcggcagtagcgcggtggtcccacctgaccccatgc
cgaactcagaagtgaaacgccgtagcgccgatggtagtgtggggtctccc
catgcgagagtagggaactgccaggcatcaaataaaacgaaaggctcagt
cgaaagactgggcctttcgttttatctgttgtttgtcggtgaacgctctc
ctgagtaggacaaatccgccgggagcggatttgaacgttgcgaagcaacg
gcccggagggtggcgggcaggacgcccgccataaactgccaggcatcaaa
ttaagcagaaggccatcctgacggatggcctttttgcgtttctacaaact
cttttgtttatttttctaaatacattcaaatatgtatccgctcatgagac
aataaccctgataaatgcttcaataatattgaaaaaggaagagtatgagt
attcaacatttccgtgtcgcccttattcccttttttgcggcattttgcct
tcctgtttttgctcacccagaaacgctggtgaaagtaaaagatgctgaag
atcagttgggtgcacgagtgggttacatcgaactggatctcaacagcggt
aagatccttgagagttttcgccccgaagaacgttttccaatgatgagcac
ttttaaagttctgctatgtggcgcggtattatcccgtgttgacgccgggc
aagagcaactcggtcgccgcatacactattctcagaatgacttggttgag
tactcaccagtcacagaaaagcatcttacggatggcatgacagtaagaga
attatgcagtgctgccataaccatgagtgataacactgcggccaacttac
actgacaacgatcggaggaccgaaggagctaaccgcttttttgcacaaca
tgggggatcatgtaactcgccttgatcgttgggaaccggagctgaatgaa
gccataccaaacgacgagcgtgacaccacgatgcctgtagcaatggcaac
aacgttgcgcaaactattaactggcgaactacttactctagcttcccggc
aacaattaatagactggatggaggcggataaagttgcaggaccacttctg
cgctcggcccttccggctggctggtttattgctgataaatctggagccgg
tgagcgtgggtctcgcggtatcattgcagcactggggccagatggtaagc
cctcccgtatcgtagttatctacacgacggggagtcaggcaactatggat
gaacgaaatagacagatcgctgagataggtgcctcactgattaagcattg
gtaactgtcagaccaagtttactcatatatactttagattgatttaaaac
ttcatttttaatttaaaaggatctaggtgaagatcctttagataatctca
tgaccaaaatcccttaacgtgagttttcgttccactgagcgtcagacccc
gtagaaaagatcaaaggatcttcttgagatcctttttttctgcgcgtaat
ctgctgcttgcaaacaaaaaaaccaccgctaccagcggtggtttgtttgc
cggatcaagagctaccaactctttttccgaaggtaactggcttcagcaga
gcgcagataccaaatactgtccttctagtgtagccgtagttaggccacca
cttcaagaactctgtagcaccgcctacatacctcgctctgctaatcctgt
taccagtggctgctgccagtggcgataagtcgtgtcttaccgggttggac
tcaagacgatagttaccggataaggcgcagcggtcgggctgaacgggggg
ttcgtgcacacagcccagcttggagcgaacgacctacaccgaactgagat
acctacagcgtgagctatgagaaagcgccacgcttcccgaagggagaaag
gcggacaggtatccggtaagcggcagggtcggaacaggagagcgcacgag
ggagcttccagggggaaacgcctggtatctttatagtcctgtcgggtttc
gccacctctgacttgagcgtcgatttttgtgatgctcgtcaggggggcgg
agcctatggaaaaacgccagcaacgcggcctttttacggttcctggcctt
ttgctggccttttgctcacatgttctttcctgcgttatcccctgattctg
tggataaccgtattaccgcctttgagtgagctgataccgctcgccgcagc
cgaacgaccgagcgcagcgagtcagtgagcgaggaagcggaagagcgcct
gatgcggtattttctccttacgcatctgtgcggtatttcacaccgcatat
ggtgcactctcagtacaatctgctctgatgccgcatagttaagccagtat
acactccgctatcgctacgtgactgggtcatggctgcgccccgacacccg
ccaacacccgctgacgcgccctgacgggcttgtctgctcccggcatccgc
ttacagacaagctgtgaccgtctccgggagctgcatgtgtcagaggtttt
caccgtcatcaccgaaacgcgcgaggcagcagatcaattcgcgcgcgaag
gcgaagcggcatgcataatgtgcctgtcaaatggacgaagcagggattct
gcaaaccctatgctactccgtcaagccgtcaattgtctgattcgttacca
attatgacaacttgacggctacatcattcactttttcttcacaaccggca
cggaactcgctcgggctggccccggtgcattttttaaatacccgcgagaa
atagagttgatcgtcaaaaccaacattgcgaccgacggtggcgataggca
tccgggtggtgctcaaaagcagcttcgcctggctgatacgttggtcctcg
cgccagcttaagacgctaatccctaactgctggcggaaaagatgtgacag
acgcgacggcgacaagcaaacatgctgtgcgacgctggcgatatcaaaat
tgctgtctgccaggtgatcgctgatgtactgacaagcctcgcgtacccga
ttatccatcggtggatggagcgactcgttaatcgcttccatgcgccgcag
taacaattgctcaagcagatttatcgccagcagctccgaatagcgccctt
ccccttgcccggcgttaatgatttgcccaaacaggtcgctgaaatgcggc
tggtgcgcttcatccgggcgaaagaaccccgtattggcaaatattgacgg
ccagttaagccattcatgccagtaggcgcgcggacgaaagtaaacccact
ggtgataccattcgcgagcctccggatgacgaccgtagtgatgaatctct
cctggcgggaacagcaaaatatcacccggtcggcaaacaaattctcgtcc
ctgatttttcaccaccccctgaccgcgaatggtgagattgagaatataac
ctttcattcccagcggtcggtcgataaaaaaatcgagataaccgttggcc
tcaatcggcgttaaacccgccaccagatgggcattaaacgagtatcccgg
cagcaggggatcattttgcgcttcagccatacttttcatactcccgccat
tcagag

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

### REFERENCES

The following references are provided to aid in understanding the invention and are incorporated herein by reference to the extent permitted by applicable statute and regulation.
1. Biery M. C., Stewart F. J., Stellwagen A. E., Raleigh E. A., Craig N. L., “A simple in vitro Tn7-based transposition system with low target site selectivity for genome and gene analysis”. Nucleic Acids Res. 2000 Mar. 1, 28(5):1067-77.
2. Goryshin I. Y., Reznikoff W. S., “Tn5 in vitro transposition”., J Biol Chem. 1998 Mar. 27; 273(13):7367-74
3. Haapa S, Suomalainen S, Eerikainen S, Airaksinen M, Paulin L, Savilahti H. “An efficient DNA sequencing strategy based on the bacteriophage mu in vitro DNA transposition reaction.” Genome Res. 1999 Mar., 9(3):308-15
4. O'Brochta D. A., Warren W. D., Saville K. J., Atkinson P. W., “Henries, a functional non-Drosophilid insect gene vector from Musca domestica”. Genetics. 1996 March; 142(3):907-14
5. Zhou L, Mitra R, Atkinson P. W., Hickman A B, Dyda F, Craig N. L., “Transposition of hAT elements links transposable elements and V(D)J recombination”. Nature. 2004 Dec. 23; 432(7020):995-1001.
6. Perez Z. N., Musingarimi P, Craig N. L., Dyda F, Hickman A. B.,” Purification, crystallization and preliminary crystallographic analysis of the Hermes transposase. Acta Crystallogr Sect F Struct Biol Cryst Commun 2005 Jun. 1; 61(Pt 6):587-90
7. Hickman A B, Perez Z. N., Zhou L., Musingarimi P., Ghirlando R., Hinshaw J. E., Craig N. L., Dyda F., “Molecular architecture of a eukaryotic DNA transposase. Nat Struct Mol Biol. 2005 August; 12(8):715-21
8. Gangadharan S., Mularoni L., Fain-Thornton J., Wheelan S. J., Craig N. L., “DNA transposon Hermes inserts into DNA in nucleosome-free regions in vivo”. Proc Natl Acad Sci USA. 2010 Dec. 21; 107(51):21966-72.
9. Zhou L, Mitra R, Atkinson P. W., Hickman A B, Dyda F, Craig N. L. “Transposition of hAT elements links transposable elements and V(D)J recombination”. Nature 2004 Dec. 23; 432(7020):995-1001.
10. Hickman A B, Perez Z. N., Zhou L., Musingarimi P., Ghirlando R., Hinshaw J. E., Craig N. L., Dyda F. “Molecular architecture of a eukaryotic DNA transposase”. Nat Struct Mol Biol. 2005 August; 12(8):715-21.

### Other Embodiments

While the invention has been described in conjunction with the detailed description thereof, the foregoing description is intended to illustrate and not limit the scope of the invention, which is defined by the scope of the appended claims Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. An improved hyperactive mutant transposase comprising a sequence selected from the group consisting of SEQ ID NO: 21, SEQ ID NO: 22, SEQ ID NO: 23, and SEQ ID NO: 24.

2. An improved hyperactive transposase comprising a mutation selected from the group consisting of E55K, A290S, G366E, G366W, I370V, F433S, E459G, I498T, S499L, and T500M when numbered in accordance with HermesI-612 wildtype (SEQ ID NO: 21).

3. A method of fragmenting and tagging target DNA sequences comprising the steps of:
providing ligand labeled Hermes LEs;
reacting the labeled Hermes LEs with an improved mutant transposase of claim 1 and target DNA sequences whereby each target DNA sequence becomes fragmented and each DNA fragment is labeled at either end by one of the labeled Hermes LEs;
purifying the labeled DNA fragments using an affinity system that binds the ligand.

4. The method according to claim 3, wherein the affinity system employs beads that bind the ligand.

5. The method according to claim 3, wherein the beads are magnetic beads.

6. The method according to claim 3, further comprising a step of using a DNA polymerase to fill in gaps.

7. The method according to claim 6, wherein the DNA polymerase is T4 polymerase.

8. The method according to claim 3, wherein the ligand is biotin or polyhistidine of at least six histidine residues and the affinity system is biotinstreptavidin or nickel or cobalt affinity material, respectively.

9. The method according to claim 3, further comprising a step of enzymatically cutting the tagged DNA following the step of purifying to replace one of the labeled Hermes LEs on each fragment with a specific terminal sequence.

10. The method according to claim 9, wherein PCR, DNA ligase or DNA polymerase chain extension is used to add the specific terminal sequence.

11. The method according to claim 3, further comprising the step of using a second transposon system to introduce a second tag into each DNA fragment.

12. The method according to claim 11, wherein the step of using a second transposon system follows the step of purifying.

13. The method according to claim 12, wherein the second transposon system is a piggy Bac transposon.

14. A method of fragmenting and tagging target DNA sequences comprising the steps of:
providing tagged Hermes LEs bearing at least one specific sequence tag; and
reacting the tagged Hermes LEs with an improved mutant transposase of claim 1 and target DNA sequences whereby each target DNA sequence becomes fragmented and each DNA fragment is labeled at either end by one of the tagged Hermes LEs.

15. The method according to claim 14, further comprising a step of employing a DNA polymerase to fill in gaps.

16. The method according to claim 15, wherein the DNA polymerase is T4 polymerase.

17. The method according to claim 14, further comprising the step of using a second transposon system to introduce a second tag into each DNA fragment.

18. The method according to claim 17, wherein the second transposon system is a piggy Bac transposon.

19. The method according to claim 14, further comprising a step of enzymatically cutting the tagged DNA following the step of purifying to replace one of the tagged Hermes LEs on each fragment with a specific terminal sequence.

20. The method according to claim 19, wherein PCR, DNA ligase or DNA polymerase chain extension is used to add the specific terminal sequence.
